(19) United States
(12) Patent Application Publication
(10) Pub. No.: US 2003/0216761 A1
(43) Pub. Date: Nov. 20, 2003

(54) Guidewire system

(76) Inventor: Samuel Shiber, Manchester, NH (US)

Correspondence Address: SAMUEL SHIBER, 365 KEARNEY CR, MANCHESTER, NH, 03104, US

(21) Appl. No.: 10/463,189

(22) Filed: Jun. 17, 2003

Related U.S. Application Data
(63) Continuation-in-part of application No. 10/172,036, filed on Jun. 14, 2002.
Application No. 10/172,036 is a continuation-in-part of application No. 09/643,181, filed on Aug. 21, 2000, now Pat. No. 6,440,148.
Application No. 09/643,181 is a continuation-in-part of application No. 09/286,218, filed on Apr. 5, 1999, now Pat. No. 6,106,538.
Application No. 09/286,218 is a continuation-in-part of application No. 08/904,972, filed on Aug. 1, 1997, now abandoned.
Application No. 08/904,972 is a continuation-in-part of application No. 08/516,772, filed on Aug. 18, 1995, now Pat. No. 5,653,696.
Application No. 08/516,772 is a continuation-in-part of application No. 08/107,453, filed on Aug. 17, 1993, now Pat. No. 5,443,443.
Application No. 08/107,453 is a continuation-in-part of application No. 07/913,231, filed on Jul. 14, 1992, now Pat. No. 5,334,211.
Application No. 07/913,231 is a continuation-in-part of application No. 07/662,558, filed on Feb. 28, 1991, now Pat. No. 5,306,244.
Application No. 07/662,558 is a continuation-in-part of application No. 07/499,726, filed on Mar. 27, 1990, now Pat. No. 5,135,531.

(51) Int. Cl. A61B 17/22

(52) U.S. Cl. 606/159

(57) ABSTRACT

A flexible guidewire system, for crossing an obstruction located in a patient's vessel, comprising a flexible pilot wire and a flexible tubular casing slidable thereon, at least a distal portion of the casing being a helical wire that is gated at its distal end, and a coupling means for connecting the casing to a drive means.

### CROSS REFERENCE TO RELATED APPLICATIONS

This application is a continuation in part (CIP) of co-pending application Ser. No. 10/172,036 filed Jun. 14, 2002 (CT21) which is CIP of application Ser. No. 09/643,181 filed Aug. 21, 2000 (CT20 now U.S. Pat. No. 6,440,148) which is a CIP of application Ser. No. 09/286,218 filed Apr. 15, 1999 (CT19 now U.S. Pat. No. 6,106,538) which is a CIP of application Ser. No. 08/904,972 filed Aug. 11, 1997 (CT18 abandoned) which is a CIP of application Ser. No. 08/516,772 filed Aug. 18, 1995 (CT17 now U.S. Pat. No. 5,653,696) which is a CIP of application Ser. No. 08/107,453 filed Aug. 17, 1993 (CT16 now U.S. Pat. No. 5,443,443) which is a CIP of application Ser. No. 07/913,231 filed Jul. 14, 1992 (CT15 now U.S. Pat. No. 5,334,211) which is a CIP of application Ser. No. 07/662,558 filed Feb. 28, 1991 (CT14 now U.S. Pat. No. 5,306,244) which is a CIP of application Ser. No. 07/499,726 filed Mar. 27, 1990 (CT13 now U.S. Pat. No. 5,135,531).

All of the above are being incorporated herein by reference.

### BACKGROUND AND OBJECTIVES OF THE INVENTION

With age a large percentage of the population develops atherosclerotic and thrombotic obstructions resulting in partial or total occlusions of blood vessels in various parts of the human anatomy. Such obstructions are often treated with angioplasty or atherectomy catheters and a common preparatory step to such procedures is the insertion of a guidewire across the obstruction.

An objective of the present invention is to provide a simple and reliable flexible guidewire system capable crossing tortuous vasculature and obstructions, particularly tight and total obstructions.

The above and other objectives of the invention will become apparent from the following discussion and the accompanying drawings.

### BRIEF DESCRIPTION OF THE FIGURES

FIG. 1 schematically shows a flexible guidewire system for crossing an obstruction in a vessel. The system is inserted at the patient's groin area, through the arterial system of the patient, into his obstructed coronary artery (the anatomy and system are not drawn to scale).

FIG. 2 shows a cross sectioned view of a flexible guidewire system with a casing in the form of a helical wire where the spacing between its distal coils is gated by a short tube and its proximal coils are attached to a coupling means for connecting the casing to a drive means. A pilot wire, comprising a hollow tube through which an inflatable chamber that is attached to its distal end section can be inflated and deflated, serves as a guidewire over which the casing can be slid and rotated.

FIG. 3 shows same embodiment as in FIG. 2 wherein a standard guidewire serves as the pilot wire.

FIG. 4 shows an enlarged partially cross sectioned view (along line 4-4 marked on FIG. 5) of the distal end section of the casing that is shown in FIGS. 2 and 3.

FIG. 5 shows an end view of the helical wire shown in FIG. 4.

FIG. 6. shows a casing wherein the distal end of the flexible casing is gated by closely wound coils of the helical wire and the midsection of the flexible casing is made of distantly wound coils made from a wire that is a continuation of the wire of which the distal end is made. The wire has a round cross section and the casing is tubular, i.e., it defines a continuous lumen in which the pilot wire is nested and can extend from either end of the casing.

FIG. 6A shows an optional distal end section of the casing shown in FIG. 6 that is curved.

FIG. 7 shows an end view of the helical wire shown in FIG. 6.

FIG. 8. shows a casing similar to the one shown in FIG. 6 except that the helical wire has a flattened cross section.

FIG. 9 shows an end view of the helical wire shown in FIG. 8.

FIGS. 10, 11 and 12 show optional cross sections of flattened wires.

FIG. 13 shows a partially cross sectioned view of a system with an inflatable chamber located at the distal end of a flexible sleeve.

FIG. 14 shows a cross sectioned view of the system shown in FIG. 13, along a line 14-14 marked on FIG. 13.

FIG. 15 shows a partially cross sectioned view of a system with a flexible sleeve having a selectively actuatable tongue at its distal end.

FIG. 16 shows a cross sectioned view of the system shown in FIG. 15 along the line 16-16 marked on FIG. 15.

### DETAILED DESCRIPTION OF THE DRAWINGS

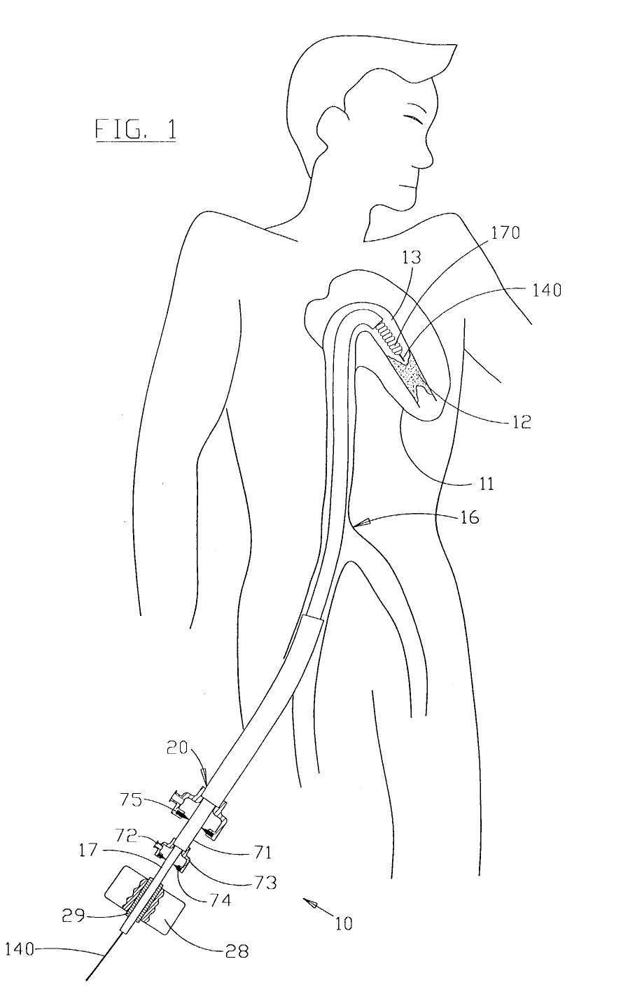

FIG. 1 generally shows a flexible guidewire system 10 for crossing an obstruction 12 located in a patient's coronary vessel 13 serving the heart 11. The system is introduced through the skin into the patient's arterial system through a flexible sleeve 71 that isolates it from the arteries' walls and directs the system to the obstruction site. A nipple 72 is connected to the flexible sleeve through an annular chamber 73 that is attached to the proximal end of the sleeve. The chamber is equipped with a seal 74 which seals around a coupling means 17 and communicates fluid entering a nipple 72 through the sleeve into the vessel. Optionally, the distal end section of the sleeve is curved, as shown, to direct the system into the vessel and selectively bias it in the vessel. The sleeve 71 can be inserted into the vasculature through a standard introducer 20 (standard introducers are sold by numerous companies, e.g., TFX Medical, Jaffrey, NH or Boston Scientific, Natick, Mass.).

The system comprises elongated nested parts that can rotate and slide one relative to the other, and their ends which goes further into the vessel shall be referred to as “distal” and their other ends shall be referred to as “proximal”.

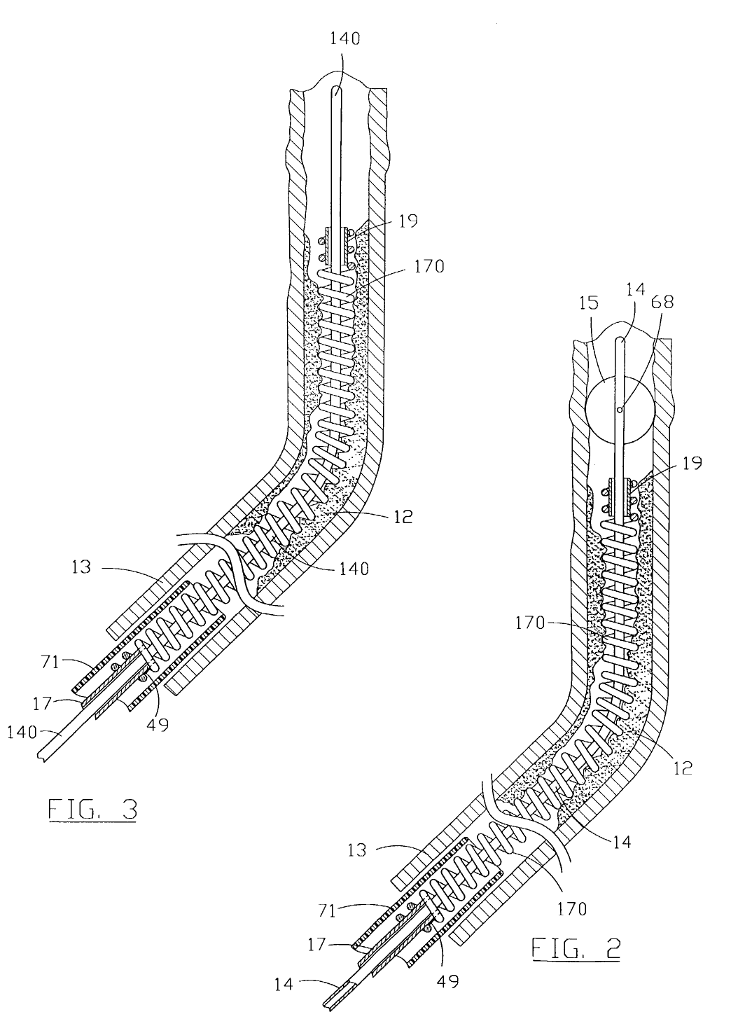

FIG. 2 shows the system 10 which comprises a flexible pilot wire 14 and a flexible casing slidable thereon. The casing comprises a helical wire 170 that is gated at its distal end by a tube section 19 that is attached to the helical wire and closes the spacing between its distal coils. Thus the gated distal end of the helical wire keeps the pilot wire inside the helical wire's lumen 21 (note FIG. 5) by preventing the pilot wire from working its way between the coils particularly while the helical wire is rotated. The pilot wire 14 is hollow and is equipped with an inflatable chamber 15 that is attached to its distal end. The chamber 15 can be inflated and deflated through the hollow flexible pilot wire and an orifice 68 to center the flexible pilot wire in the vessel, to cushion the contact between the flexible pilot wire and the vessel wall, as well as for anchoring it to the vessel wall (similar parts will be indicated by same numbers throughout the FIGURES).

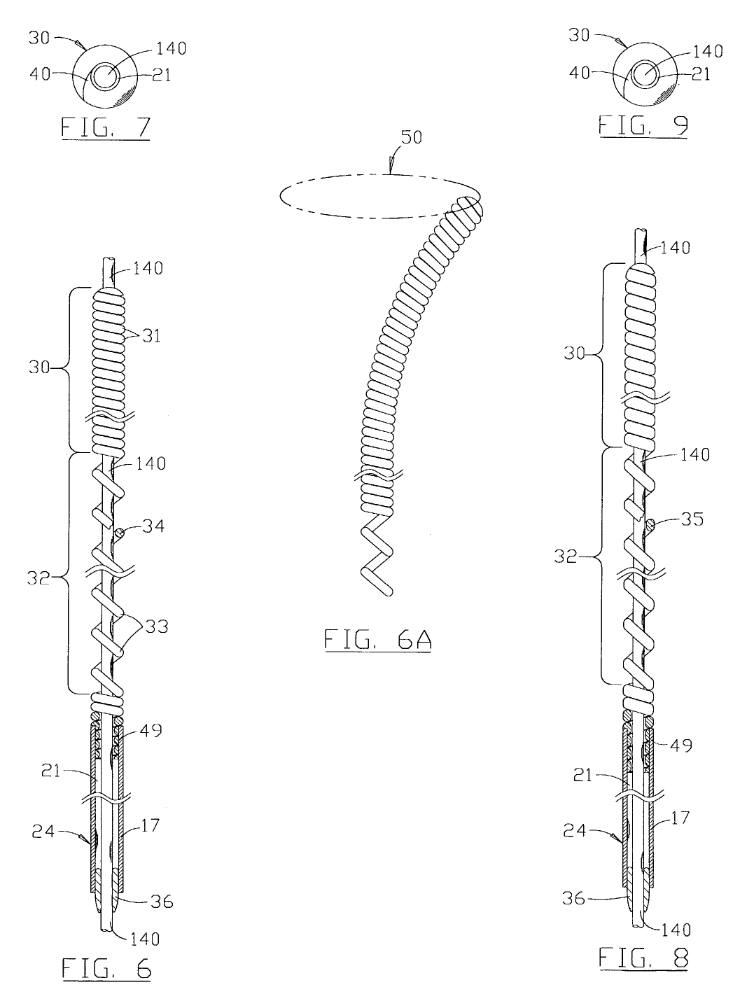

Coupling means in the form of a tube 17 is attached to a proximal end of the helical wire by a weld. 49 for connecting the casing to a drive means that can linearly advance and rotate the casing over the pilot wire in the vessel. To facilitate the linear motion and rotation, the tube 17 has a smooth outside surface 24 (note FIGS. 6 and 8) that allows it to slide through a seal 74 (note FIG. 1) and rotate without excessive leakage, or if the introducer 20 is used alone without a sleeve, through a seal of the introducer 75. A physician can rotate and linearly drive the coupling means with his fingers. Alternatively, an optional motor 28 (note FIG. 1) can be used to provide the rotation through its hollow output shaft 29 that is slid over and frictionally engages the coupling means 17 while the linear motion is provided manually by the physician's hand that holds and moves the motor.

FIG. 3 shows a flexible guidewire system wherein the flexible pilot wire is constructed like a standard flexible guidewire 140 (standard guidewires are sold by numerous companies, e.g., TFX Medical or Boston Scientific).

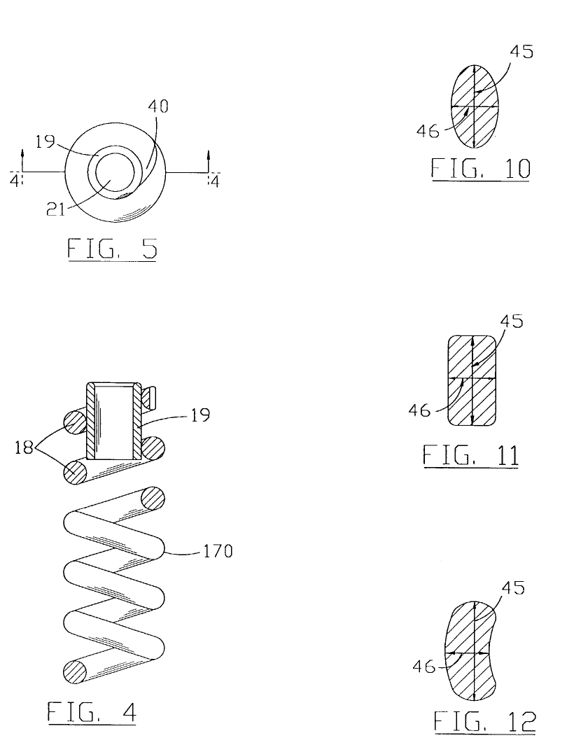

FIG. 4 shows an enlarged, partially sectioned view (along line 4-4 marked on FIG. 5) of the distal end section of the helical wire 170 where the distal entry to the helical wire is gated by the tube 19, preferably made from radio opaque material (for example an alloy comprising gold and/or platinum), attached to the internal diameter of the casing that closes the spacing between its distal coils 18 and keeps the pilot wire inside the casing's lumen 21 (note FIG. 5).

FIG. 5 shows a distal end view of the casing shown in FIG. 4 having a pointed distal end tip 40, adjacent to the tube 19, to ease penetration into the obstruction material. The tip 40 can be manufactured by gradually grinding down the wire to form a smooth inclined plane minimizing trauma that it may cause to the vasculature 16 and the vessel 13.

FIG. 6 shows a flexible guidewire system where the distal end of the casing is gated by closely wound coils 31 of a helical wire 30. The closely wound coils prevent the pilot wire from working its way between the coils when the helical wire is rotated. It also prevents the pilot wire from exiting the helical wire's lumen 21 (note FIG. 7) when the casing is advanced beyond the distal tip of the pilot wire and then the pilot wire is advanced beyond the distal tip of the casing. In addition, the closely wound coils make the distal end section of the casing more flexible and more radio-opaque. The midsection of the casing 32 comprises distantly wound coils 33 that are preferably made of a continuation of the same wire 34 of which the distal section of the casing is made. The wire 34 has a round cross section. The distantly wound coils provide increased torsional and longitudinal rigidity and thereby reduce the elastic angular and linear deformation between the distal and proximal end of the casing under torque and tension, respectively.

Coupling means 17, attached to a proximal end of the midsection of the casing, has a seal 36 at its proximal end to allow the guidewire 140 to slide and rotate relative to the casing while maintaining a seal around it. The casing is tubular, i.e., it defines a continuous lumen 21, in which the pilot wire is nested, that extends through the casing and allows the pilot wire to extend from either end (it should be understood that the seal 36 may close in the absence of the guidewire).

Optionally a distal end section of the casing is curved, as shown in FIG. 6A, so that, as the casing is rotated in order to start penetrating the obstruction, the distal tip moves along a circular pass 50, increasing the probability that it would locate a softer point of the obstruction.

FIG. 7 shows an end view of the system shown in FIG. 6.

FIG. 8. shows a flexible guidewire system similar to the one shown in FIG. 6 except that the wire 35 has a flattened cross section and it is wound on its side, as discussed below.

FIG. 9 shows an end view of the system shown in FIG. 8.

FIGS. 10, 11 and 12 illustrate examples of flattened-wires (the term “flattened-wire”, as used in this application, is derived from a common method of manufacturing such wire by flattening a wire with a round cross section between two adjacent rollers). The flattened-wires have a non-round cross section with a long-axis 45, a short-axis 46, and as used in this application, the term “wound on its side” refers to the wire wound with its long-axis being approximately parallel to the helical wire's longitudinal axis.

FIGS. 13 and 14 show side and end views, respectively, of a partially cross sectioned biasing means in the form of an asymmetrical inflatable chamber 81 formed at the distal end of a flexible deflecting sleeve 82 which, when inflated through a channel 83 formed in the sleeve's wall, bears against the vessel's wall, eccentrically biasing the flexible sleeve in the vessel. When deflated, the chamber conforms to the sleeve to minimize interference with its insertion into the vessel. Alternatively, the chamber can be shaped as an asymmetrical toroidal inflatable chamber 81′ as shown in FIG. 14 by interrupted lines. This chamber, when inflated, establishes peripheral contact with the vessel's wall and thereby blocks blood flow between the sleeve and the vessel wall as well as eccentrically biasing the sleeve (it can be understood that a symmetrical toroidal chamber can be provided for the purpose of blocking the flow around the sleeve while centering the biasing sleeve).

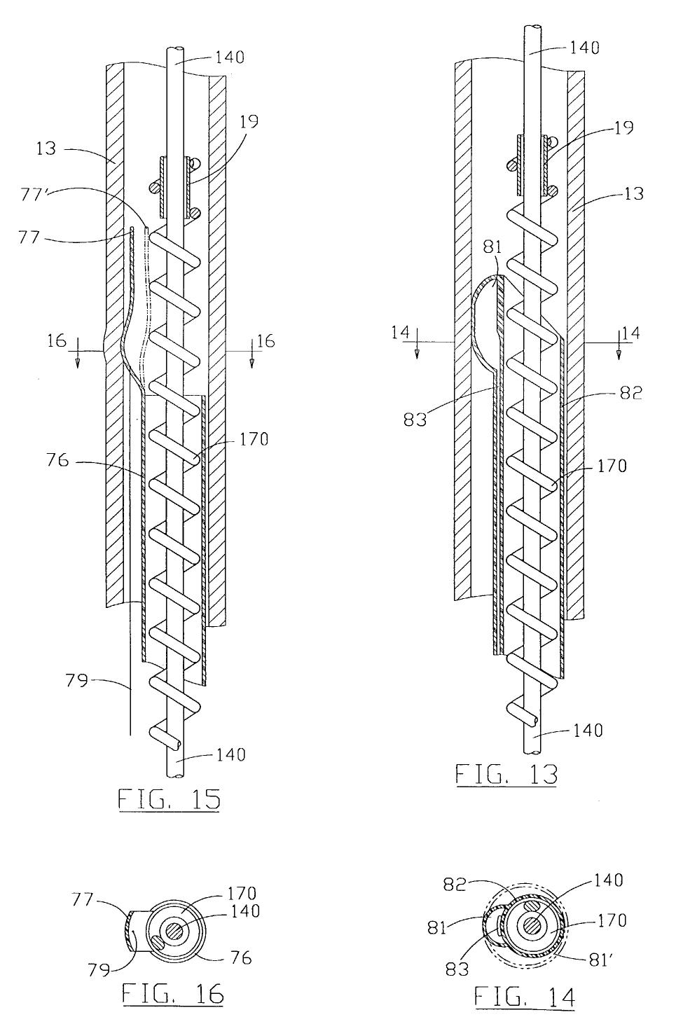

FIGS. 15 and 16 show side and end views, respectively, of a partially cross sectioned flexible sleeve 76 that has a tongue 77 which can be used to bias the sleeve in the vessel. The tongue can be energized against the vessel wall by tensioning a flexible rope 79, moving the tongue from its relaxed position which is shown by a phantom line in FIG. 15 and marked 77′ to the position shown in solid lines and marked 77.

### Operation

FIGS. 1 and 2 illustrate systems, according to the present invention, where a distal portion of the flexible pilot wire is inserted into a curved vessel, and assumes the vessel's geometry. Then a casing, preferably in the form of a helical wire, is inserted through the vasculature over the flexible pilot wire. The casing can be rotated to assist it in advancing over the pilot wire and through curves of the vasculature while the flexible pilot wire safely guides the advancing helical wire 170 through the curved vessel. It should be noted that the rotation of the casing substantially reduces the longitudinal friction between the casing and the guidewire that is nested in its lumen (assuming that the guidewire is held stationary) as well as the longitudinal friction between the casing and its surroundings, i.e., the sleeve (assuming a sleeve is used) and the vessel or vessels through which the casing is advanced. Further, if a casing in the form of a helical wire is turned in the direction that the coils are wound the rotation is translated to a force that pulls and propels the casing forward through the vessels. Such pulling force generated at the distal end is significant because in order to deliver to the distal end the same amount of force by pushing through a tortuous path (as the path through the coronary vasculature is), a larger force would be required to be applied to the proximal end of the casing which may exceed the casing's columnar strength.

Once the casing is brought to an obstruction, the process of crossing the obstruction with a system according to the present invention can be done as follows:

Advancing the flexible pilot wire into the obstruction, preferably as far as it would go.

Inserting the casing to the obstruction and rotating it in the direction that the coils are wound so that the helical wire propels itself and threads itself through the obstruction. In the process, the end of the helical wire may be advanced past the distal tip of the pilot wire and then the tip of the pilot wire may be advanced past the distal tip of the casing in a leapfrog-like manner. Once the pilot wire is advanced across the obstruction, the casing may be withdrawn, by simply pulling it or by rotating it in the opposite direction to unthread it and to minimize longitudinal friction both with the pilot wire and with the surrounding of the casing, leaving the pilot wire in place preparatory to subsequent procedures such as angioplasty or atherectomy.

It is also possible to continue and rotate the casing in direction that the coils are wound while pulling it out to increase the helical wire's proximal conveyance action, especially when working in an obstruction with a slurry-like consistency such as fresh blood clots.

The sequence of inserting the system's components into the vessel may be varied. Steps may be combined to streamline the procedure or added to improve it and to customize the procedure to the individual characteristics of the obstruction and its location and to the working preferences of the medical staff. For example, the system may be introduced percutaneously through a sleeve and/or an introducer or intra-operatively, i.e., accessing vessel directly while it is exposed surgically. Additionally, a standard guiding catheter, which is either straight or curved may be used as a sleeve or as biasing means to be inserted into the vessel to assist in positioning the system's components in the obstruction site. Further, the pilot-wire and the casing can be pre-nested before they are inserted into the vessel.

Further, a system according to the present invention can have different diameters and lengths depending on the size and site of vessel that it is intended for and on whether the system is to be used percutaneously or intra-operatively. For example, a system that is intended to be introduced percutaneously at the groin area for crossing an obstruction in a coronary vessel may utilize for a pilot wire a standard 0.014″ (″ denotes inches) guidewire that is 118″ long and have a casing with an internal diameter of 0.020″, an outside diameter of 0.045″ and a length of 50″. If the casing is gated by a closely wound coils as shown in FIG. 6 or 8, the length of the closely wound section 31 can be 8″ and the length of the coupling means 17 can be 10″. If the system utilizes a larger pilot wire such as an 0.035″ guidewire, its diameters can be increased accordingly. If the system is used in peripheral (non-coronary) blood vessels or where direct access to the vessel is gained surgically, the system can be much shorter.

The above mentioned and other modifications and substitutions can be made in the system and in its operation within the spirit of the invention and the scope of the following claims.

I claim:

1. A flexible guidewire system, for crossing an obstruction located in a patient's vessel, comprising in combination;
a flexible pilot wire;
a flexible tubular casing slidable and rotatable over said pilot wire, at least a distal portion of said casing being a helical wire that is gated at its distal end; and
a coupling means for rotating and linearly moving said casing over said pilot wire.

2. As in claim 1, wherein said distal end of said flexible casing is gated by a tube section that is attached to said helical wire.

3. As in claim 1, wherein said distal end of said flexible casing is gated by closely wound coils of said helical wire.

4. As in claim 1, wherein a midsection of said flexible casing comprises a distantly spaced coils of said helical wire.

5. As in claim 1, wherein said distal end of said flexible casing is gated by a closely wound coils of said helical wire and the midsection of said flexible casing being a distantly wound coils of said helical wire that is a continuation of the wire of which the distal end is made.

6. As in claim 1, wherein said distal end section of said casing is curved.

7. As in claim 1, wherein said flexible pilot wire is a standard guidewire.

8. As in claim 1, wherein said flexible pilot wire comprises a hollow tube.

9. As in claim 1, wherein said flexible pilot wire comprises a hollow tube with an inflatable chamber attached to its distal end section, said chamber being inflatable through said hollow tube.

10. As in claim 1, wherein the flexible guidewire system is disposed in a sleeve with a biasing means to deflect the position of said casing in said vessel.

11. As in claim 10, wherein said biasing means comprises a sleeve with a curved distal end section.

12. As in claim 10, wherein said biasing means comprises a sleeve with a selectively inflatable chamber attached to said distal end section of said sleeve.

13. A process for crossing an obstruction in a patient's vessel comprising the following steps:
inserting through the vessel, to an obstruction, a flexible pilot wire,
advancing through the vessel, over the pilot wire, a flexible tubular casing with at least a distal portion being a helical wire having a gated distal end and a coupling means for connecting said casing to a drive means,
threading the casing through the obstruction by advancing and rotating it over the pilot wire.

14. As in claim 13, wherein a portion of said flexible pilot wire is inserted distally into said vessel and provides a lever arm to angularly align said flexible casing with said vessel.

15. A process for crossing an obstruction in a patient's vessel comprising the following steps:
inserting through the vessel, into an obstruction, a flexible pilot wire,
advancing through the vessel, over the pilot wire, a flexible tubular casing with at least a distal portion being a helical wire having a gated distal end and a coupling means for connecting said casing to a drive means,
advancing and rotating the casing beyond the distal tip of the pilot wire and threading across the obstruction,
advancing the pilot wire across the obstruction, and,
withdrawing the casing leaving the pilot wire in place.
